(19) United States
(12) Patent Application Publication
(10) Pub. No.: US 2026/0257027 A1
(43) Pub. Date: Sep. 3, 2026

(54) SYSTEMS AND METHODS FOR MONITORING PATIENT INTERFACES

(71) Applicant: RESMED PTY LTD, Bella Vista, New South Wales (AU)

(72) Inventor: Andrew Roderick BATH, Sydney (AU)

(21) Appl. No.: 19/163,770

(22) PCT Filed: Mar. 7, 2024
(86) PCT No.: PCT/AU2024/050187

§ 371 Date: Sep. 9, 2025

Related U.S. Application Data
(60) Provisional application No. 63/489,295, filed on Mar. 9, 2023.

(51) Int. Cl. A61M 16/00; A61M 16/06; A61M 16/08; A61M 16/10; A61M 16/16

(52) U.S. Cl. CPC A61M 16/0051; A61M 16/0003; A61M 16/0066; A61M 16/024; A61M 16/0672; A61M 16/0816; A61M 16/107; A61M 16/16; A61M 2016/0027; A61M 2202/0208; A61M 2205/13; A61M 2205/15; A61M 2205/18; A61M 2205/70

(57) ABSTRACT

In one form of the present technology, a system for analysing a high flow respiratory therapy, may comprise: a blower; a nasal cannula, wherein a flow of gas travels from the blower to the nasal cannula along a flow path during administration of the high flow respiratory therapy; a sensor disposed along the flow path; and a processor configured to perform steps to: receive data from the sensor; compare the data received from the sensor to a threshold value; based at least in part on the comparison, determine whether the nasal cannula is mispositioned on a patient or displaced from the patient; and upon determining the nasal cannula is mispositioned on the patient or displaced from the patient, transmit an alert indicating that the nasal cannula is mispositioned on the patient or displaced from the patient.

### 1 CROSS REFERENCE TO RELATED APPLICATION

This application claims the benefit of priority to U.S. Application No. 63/489,295, filed Mar. 9, 2023, the entire contents of which is incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device) or other Respiratory Therapy Device (RT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. For flow therapies such as nasal HFT, the patient interface is configured to insufflate the nares but specifically to avoid a complete seal. One example of such a patient interface is a nasal cannula.

### 2.2.3.2 Respiratory Therapy (RT) Device

A RT device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RT devices may also act as flow therapy devices. Examples of RT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RT device according to one or more “compliance rules”. In order to determine a patient's compliance, a provider of the RT device, such as a health care provider, may manually obtain data describing the patient's therapy using the RT device, calculate the usage over a predetermined time period, and compare with the compliance rule. Once the health care provider has determined that the patient has used their RT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

In one form of the present technology, a system for analysing a high flow respiratory therapy, may comprise: a blower; a nasal cannula, wherein a flow of gas travels from the blower to the nasal cannula along a flow path during administration of the high flow respiratory therapy; a sensor disposed along the flow path; and a processor configured to perform steps to: receive data from the sensor; compare the data received from the sensor to a threshold value; based at least in part on the comparison, determine whether the nasal cannula is mispositioned on a patient or displaced from the patient; and upon determining the nasal cannula is mispositioned on the patient or displaced from the patient, transmit an alert indicating that the nasal cannula is mispositioned on the patient or displaced from the patient.

In some forms, the processor and the blower may be disposed within a respiratory therapy device.

In aspects, the system may further comprise a tubing that is coupled to the nasal cannula at an interface, wherein the sensor is disposed at the interface.

In at least some examples, the sensor is a pressure sensor, and wherein the threshold value is an amplitude of a pressure waveform.

In forms, the threshold value may be a patient-specific threshold value.

In further forms, the patient-specific threshold value may be based on analysis of data from the sensor that is obtained during a calibration.

In aspects, the patient-specific threshold value may be generated based on machine learning.

In at least some forms, the sensor may be a first sensor and the system may further comprise a second sensor, the steps further comprising: receive data from the second sensor; and evaluate an efficacy of the respiratory therapy based at least in part on the data received from the second sensor.

In forms, the alert may be transmitted to the patient.

In aspects, the alert may include a report provided to a medical provider.

In another form, a system for analysing a high flow respiratory therapy, may comprise: a respiratory therapy device; a nasal cannula; a tubing that is configured to couple to the nasal cannula at an interface, wherein a flow of gas travels from the respiratory therapy device, through the tubing, and to the nasal cannula during administration of the high flow respiratory therapy; a pressure sensor disposed in or on the nasal cannula or at the interface; and a processor configured to perform steps to: receive data from the pressure sensor; analyze the data received from the pressure sensor; based at least in part on the analysis, determine whether the nasal cannula is mispositioned on a patient or displaced from the patient by comparing an amplitude of a pressure waveform of the received data to a threshold amplitude; and upon determining the nasal cannula is mispositioned on the patient or displaced from the patient, transmit an alert indicating that the nasal cannula is mispositioned on the patient or displaced from the patient.

In forms, the processor may be disposed within the respiratory therapy device.

In aspects, the analysis includes comparing the data received from the pressure sensor to a patient-specific threshold value.

In some forms, the patient-specific threshold value may be based on analysis of data from the pressure sensor that is obtained during a calibration.

In further forms, the patient-specific threshold value may be generated based on machine learning.

In aspects, the system may include a second sensor, the steps further comprising: receiving data from the second sensor; and evaluating an efficacy of the respiratory therapy from the respiratory therapy device based at least in part on the data received from the second sensor.

In other forms, the system may include a second sensor, and the second sensor is a different type of sensor than the first sensor.

In forms, the alert may be transmitted to the patient.

In some aspects, the alert may include a report provided to a medical provider.

In a further example, a system for analysing a high flow respiratory therapy, may comprise: a blower; a nasal cannula, wherein a flow of gas travels from the blower to the nasal cannula along a flow path during administration of the high flow respiratory therapy; a pressure sensor disposed along the flow path; and a processor configured to perform steps to: receive data from the pressure sensor; compare an amplitude of a pressure waveform of the data received from the pressure sensor to a patient-specific threshold value; based on the comparison, analyze a positioning of the nasal cannula or an aspect of a patient's breathing; and transmit an alert to the patient based on the analysis when the analysis indicates incorrect positioning of the nasal cannula or a deviation in the aspect of the patient's breathing.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RT device that may be carried by a person, e.g., around the home of the person.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

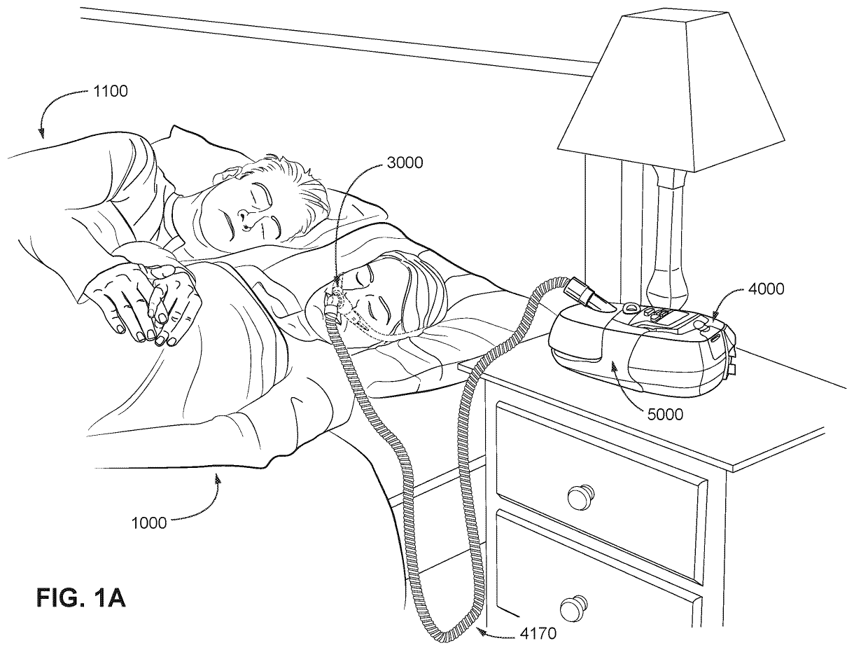

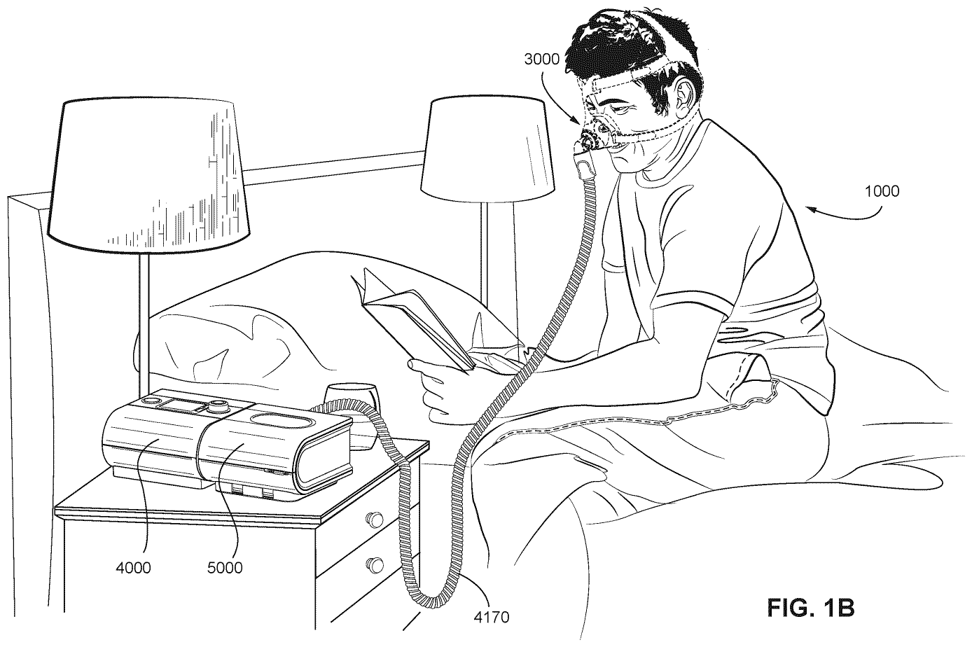

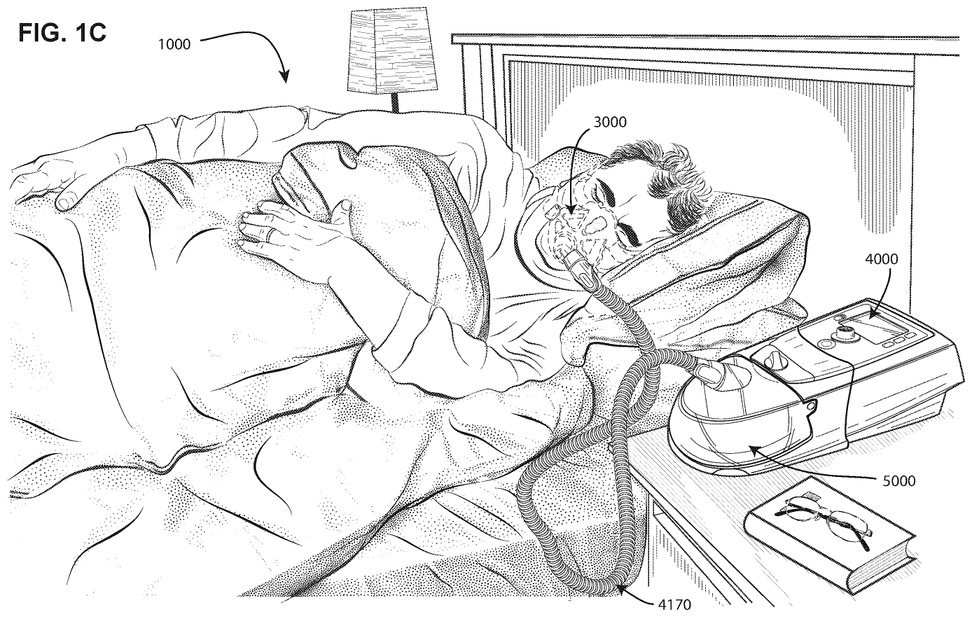

FIG. 1A shows a system including a patient 1000 wearing a patient interface 3000, receiving a supply of air (e.g., air at a positive pressure) from a respiratory therapy (“RT”) device 4000. Air from the RT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, receiving a supply of air from a RT device 4000. Air from the RT device 4000 is humidified in a humidifier 5000, and passes along an air circuit 4170 to the patient 1000.

FIG. 1C shows a system including a patient 1000 wearing a patient interface 3000, receiving a supply of air from a RT device 4000. Air from the RT device is humidified in a humidifier 5000, and passes along an air circuit 4170 to the patient 1000. The patient is sleeping in a side sleeping position.

### 4.2 Respiratory System and Facial Anatomy

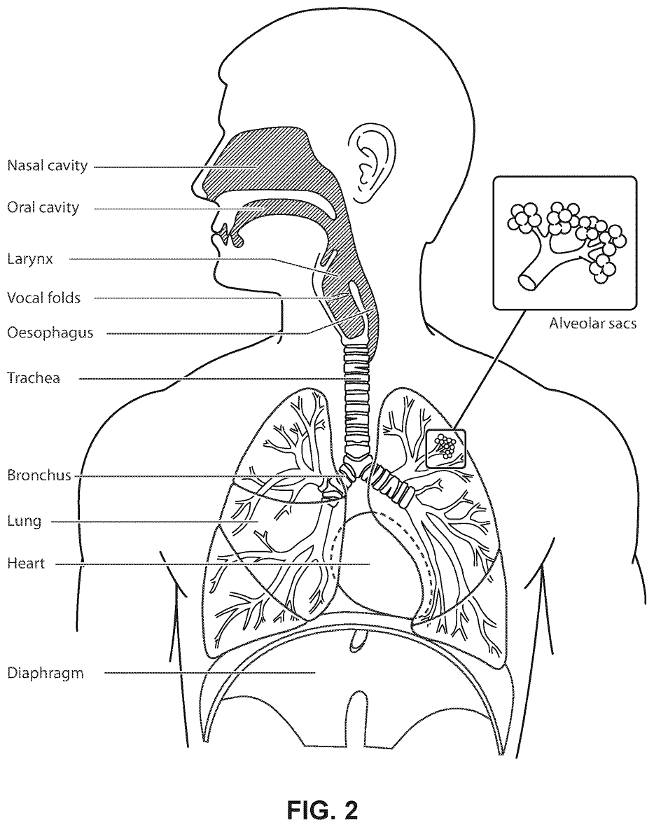

FIG. 2 shows an overview of a human respiratory system including the nasal and oral cavities, the larynx, vocal folds, oesophagus, trachea, bronchus, lung, alveolar sacs, heart and diaphragm.

### 4.3 Patient Interface

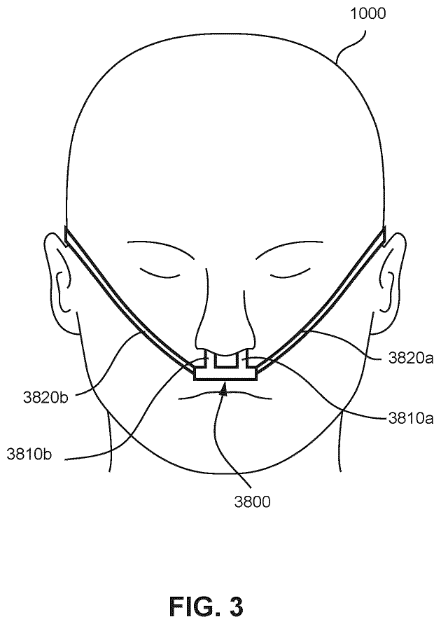

FIG. 3 shows a patient interface in the form of a nasal cannula in accordance with one form of the present technology.

### 4.4 Rt Device

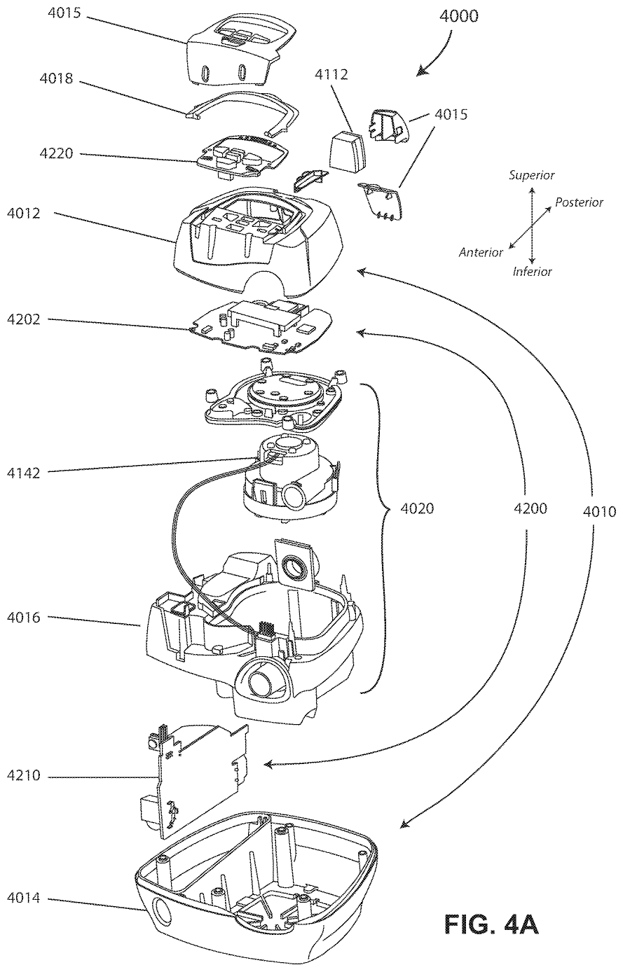

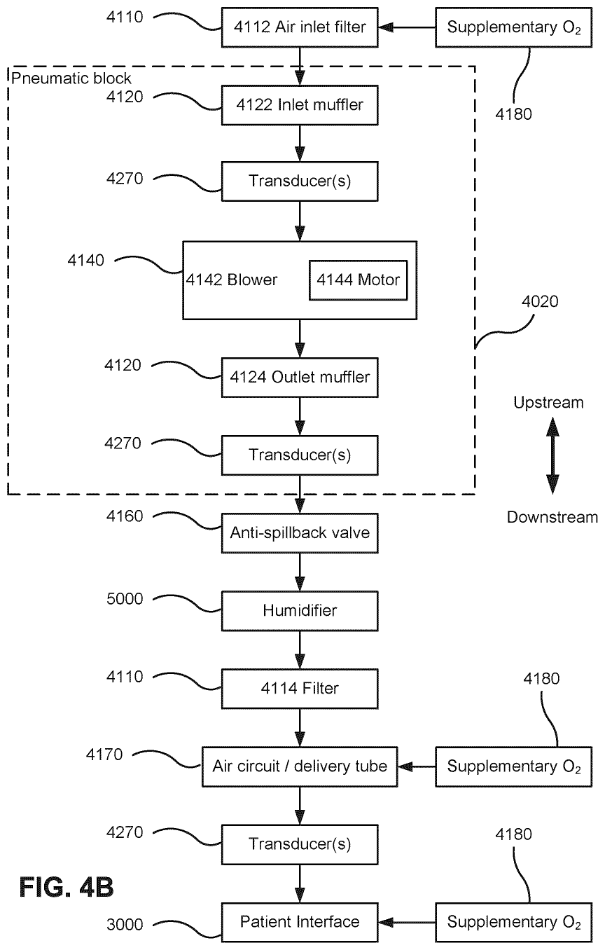

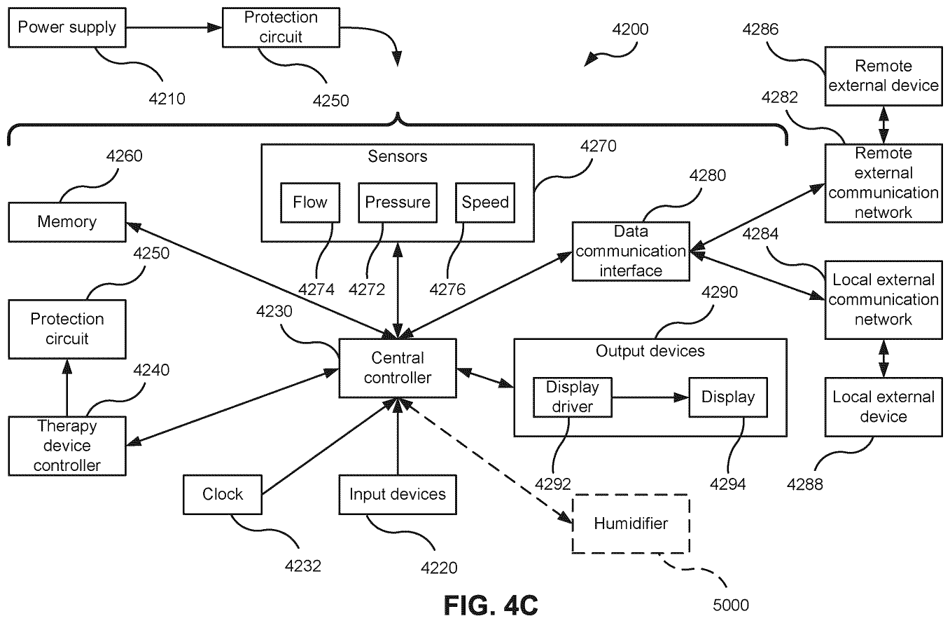

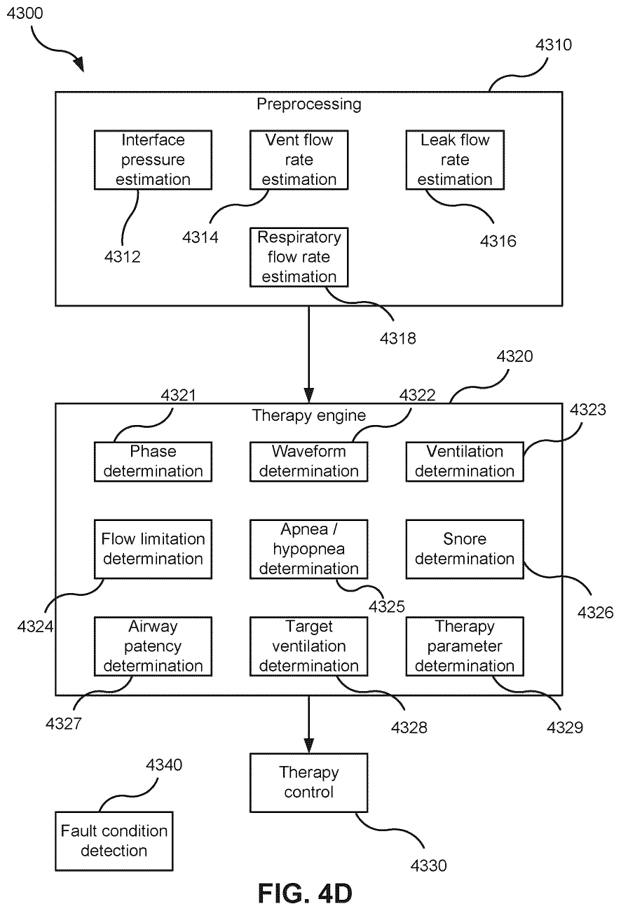

FIG. 4A shows an RT device in accordance with one form of the present technology.

FIG. 4B is a schematic diagram of the pneumatic path of an RT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RT device in accordance with one form of the present technology.

FIG. 4D is a schematic diagram of the algorithms implemented in an RT device in accordance with one form of the present technology.

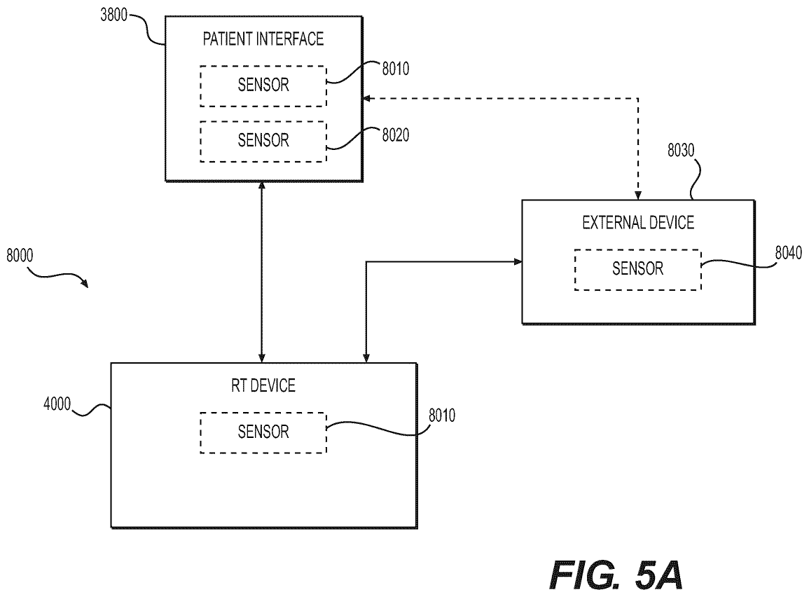

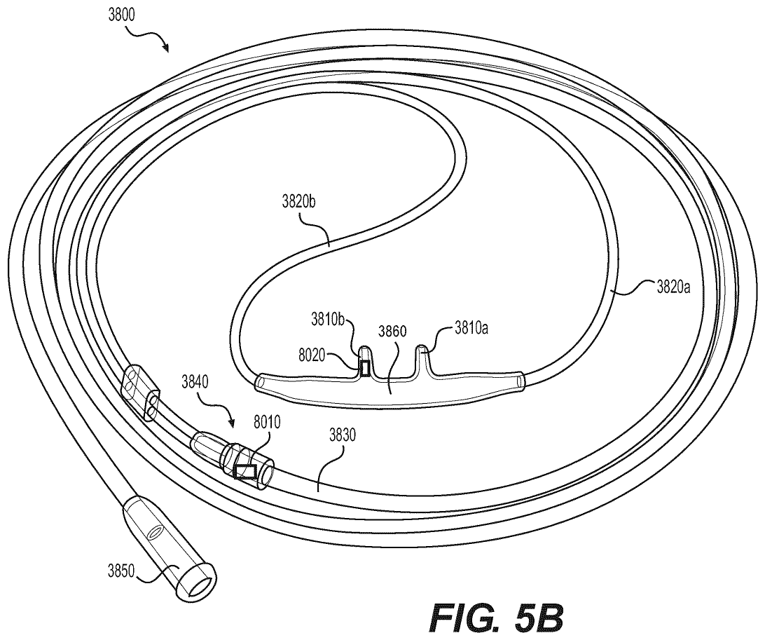

FIG. 5A depicts a system for detecting and/or communicating that a patient interface is out of position, for evaluating a therapy, or for evaluating patient breathing.

FIG. 5B depicts aspects of a patient interface for use with the system of FIG. 5A.

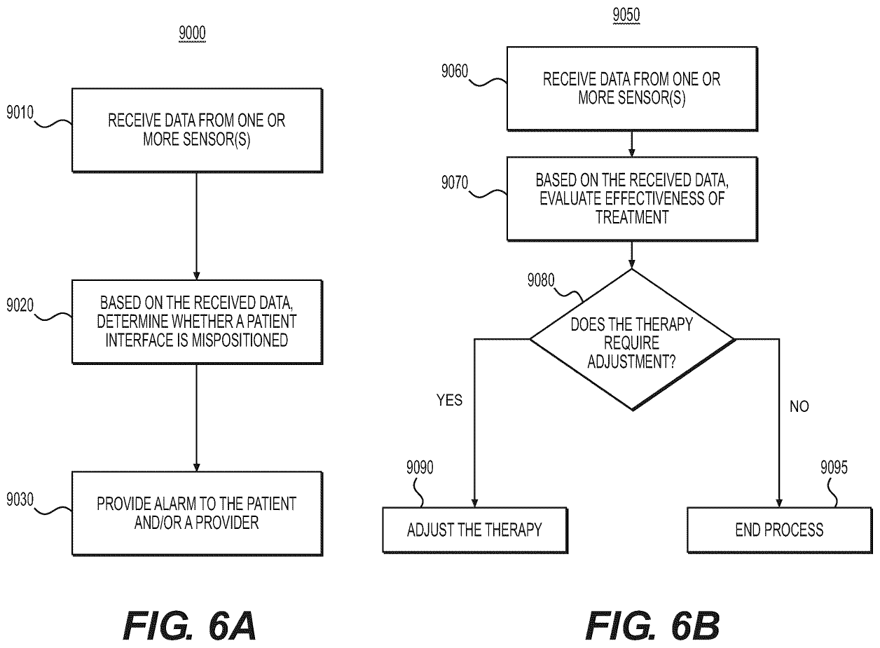

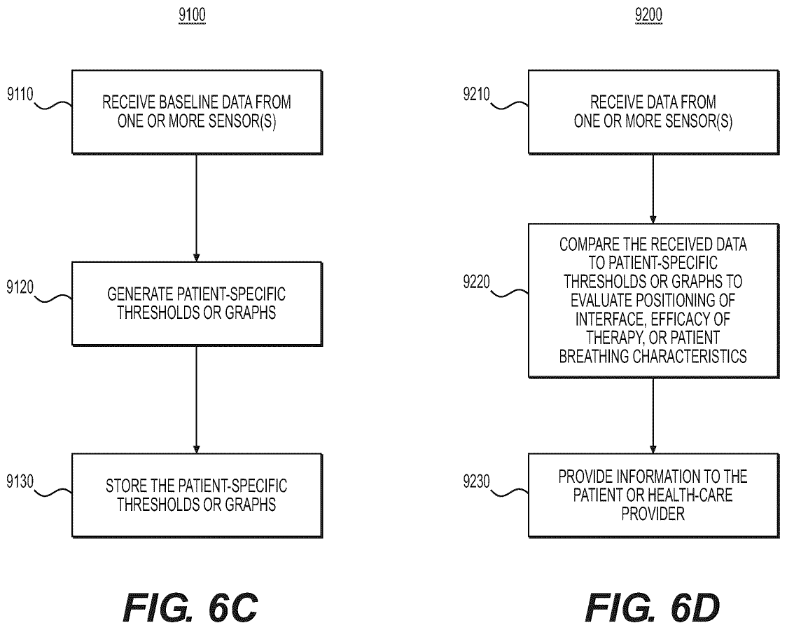

FIG. 6A shows a flow chart of a method for detecting that the patient interface is out of position.

FIG. 6B shows a flow chart of a method for evaluating and adjusting a therapy.

FIG. 6C shows a flow chart of a method for generating patient-specific thresholds for evaluation.

FIG. 6D shows a flow chart for using the patient-specific thresholds to evaluate patient interface positioning, therapy parameters, or patient breathing parameters.

### 4.5 Breathing Waveforms

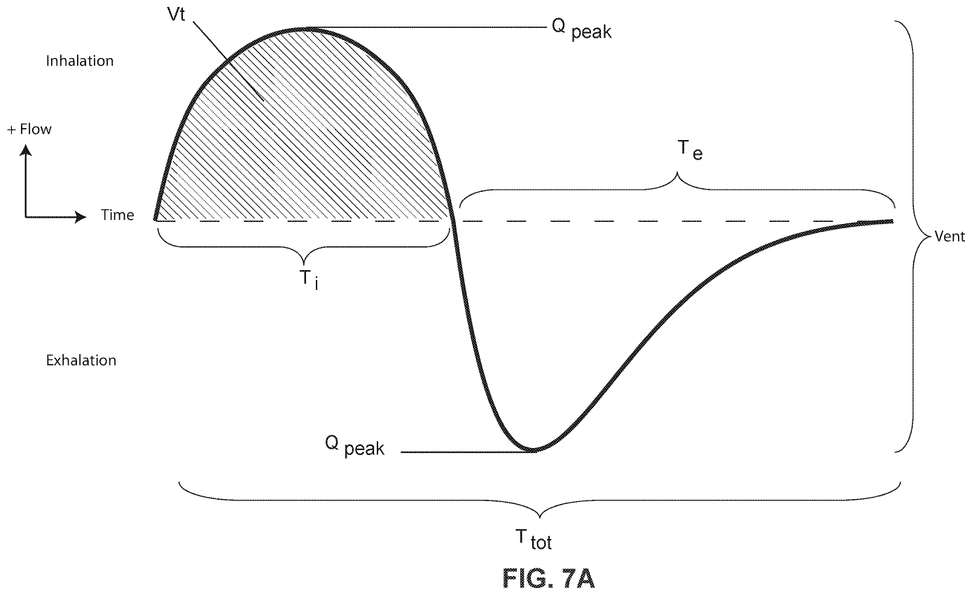

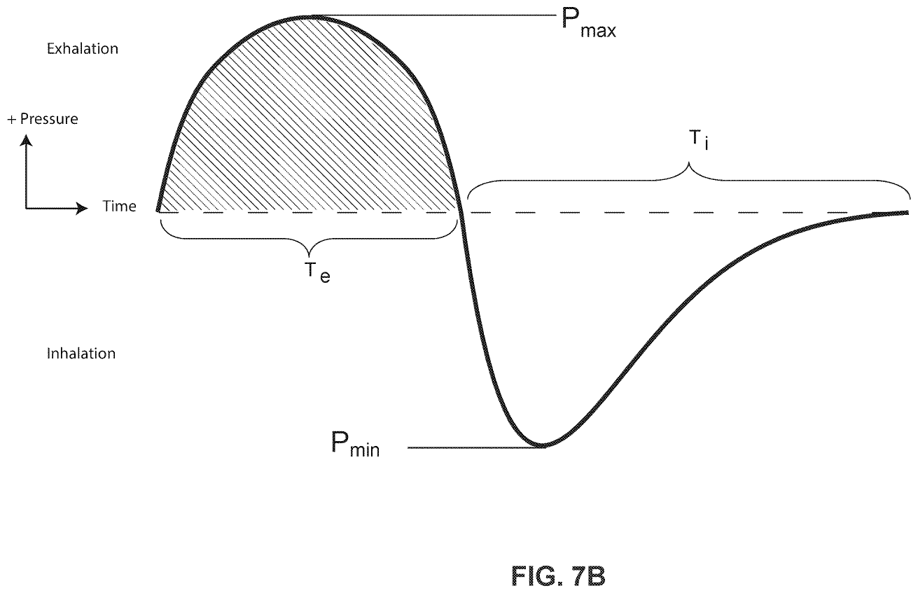

FIG. 7A shows a model typical breath waveform of a person while sleeping.

FIG. 7B shows a typical pressure waveform of a breath cycle.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise a RT device 4000 for supplying a flow of air to the patient 1000 via an air circuit 4170 and a patient interface 3000 or 3800.

### 5.3 Patient Interface

As shown in FIG. 3, an unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

### 5.4 Therapy Device

A RT device 4000 in accordance with one aspect of the present technology comprises mechanical, pneumatic, and/or electrical components and is configured to execute one or more algorithms 4300, such as any of the methods, in whole or in part, described herein. The RT device 4000 may be configured to generate a flow of air for delivery to a patient's airways, such as to treat one or more of the respiratory conditions described elsewhere in the present document.

In one form, the RT device 4000 is constructed and arranged to be capable of delivering a flow of air in a range of −20 L/min to +150 L/min while maintaining a positive pressure of at least 4 cmH2O, or at least 10cmH2O, or at least 20 cmH2O.

The RT device 4000 may have an external housing 4010, formed in two parts, an upper portion 4012 and a lower portion 4014. Furthermore, the external housing 4010 may include one or more panel(s) 4015. The RT device 4000 comprises a chassis 4016 that supports one or more internal components of the RT device 4000. The RT device 4000 may include a handle 4018.

The pneumatic path of the RT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

As shown in FIG. 4C, the RT device 4000 may have an electrical power supply 4210, one or more input devices 4220, a central controller 4230 (e.g., a processor), a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RT device 4000 may include more than one PCBA 4202.

### 5.4.1 RT Device Mechanical & Pneumatic Components

A therapy device may comprise one or more of the following components in an integral unit. In an alternative form, one or more of the following components may be located as respective separate units.

### 5.4.1.1 Air Filter(s)

A therapy device (such as the RT device 4000) in accordance with one form of the present technology may include an air filter 4110, or a plurality of air filters 4110.

In one form illustrated in FIG. 4B, an inlet air filter 4112 is located at the beginning of the pneumatic path upstream of a pressure generator 4140.

In one form illustrated in FIG. 4B, an outlet air filter 4114, for example an antibacterial filter, is located between an outlet of the pneumatic block 4020 and a patient interface 3000 or 3800.

### 5.4.1.2 Muffler(s)

An RT device in accordance with one form of the present technology may include a muffler 4120, or a plurality of mufflers 4120.

In one form of the present technology (see e.g., FIG. 4B), an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RT device, or external of the RT device. External transducers may be located for example on or form part of the air circuit, e.g., the patient interface. External transducers may be in the form of non-contact sensors such as a Doppler radar movement sensor that transmit or transfer data to the RT device.

In one form of the present technology (see e.g., FIG. 4B), one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

5.4.1.4.1 Flow rate sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.4.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.1.5 Anti-Spill Back Valve

As shown in FIG. 4B, one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.2 RT Device Electrical Components

### 5.4.2.1 Input Devices

In one form of the present technology, a RT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.2.2 Central Controller

In one form of the present technology, the central controller 4230 is one or a plurality of processors suitable to control a RT device 4000. The central controller 4230 is show in FIG. 4C.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and/or the humidifier 5000.

The central controller 4230 may be configured to provide output signal(s) to one or more of an output device 4290, a pressure generator 4140, a therapy device controller 4240, a data communication interface 4280, and/or the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with a RT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.3 Clock

The RT device 4000 may include a clock 4232 that is connected to the central controller 4230.

### 5.4.2.4 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.2.5 Protection Circuits

The one or more protection circuits 4250 in accordance with the present technology may comprise an electrical protection circuit, a temperature and/or pressure safety circuit.

### 5.4.2.6 Memory

In accordance with one form of the present technology the RT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.7 Data Communication Systems

In one form of the present technology, a data communication interface 4280 is provided, and is connected to the central controller 4230 (see e.g., FIG. 4C). Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.4.2.8 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.2.8.1 Display Driver

A display driver 4292 receives as an input the characters, symbols, or images intended for display on the display 4294, and converts them to commands that cause the display 4294 to display those characters, symbols, or images.

### 5.4.2.8.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.4.3 RT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the vent flow rate Qv, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.4.3.1.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RT device 4000 (the device flow rate Qd). The device flow rate Qd, absent any supplementary gas 4180, may be used as the total flow rate Qt. The interface pressure algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000 or 3800. The pressure, Pm, in the patient interface 3000 or 3800 may be estimated as the device pressure Pd minus the air circuit pressure drop ΔP.

### 5.4.3.1.2 Vent Flow Rate Estimation

In one form of the present technology, a vent flow rate estimation algorithm 4314 receives as an input an estimated pressure, Pm, in the patient interface 3000 or 3800 from the interface pressure estimation algorithm 4312 and estimates a vent flow rate of air, Qv, from a vent 3400 in a patient interface 3000 or 3800. The dependence of the vent flow rate Qv on the interface pressure Pm for the particular vent 3400 in use may be modelled by a vent characteristic Qv(Pm).

### 5.4.3.1.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 5.4.3.2 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.3.2.1 Phase Determination

In one form of the present technology, the RT device 4000 does not determine phase.

In one form of the present technology, a phase determination algorithm 4321 receives as an input a signal indicative of respiratory flow rate, Qr, and provides as an output a phase Φ of a current breathing cycle of a patient 1000.

In some forms, known as discrete phase determination, the phase output Q is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RT device 4000) when the respiratory flow rate Qr has a value that exceeds a positive threshold, and a discrete value of 0.5 revolutions (thereby “cycling” the RT device 4000) when a respiratory flow rate Qr has a value that is more negative than a negative threshold. The inhalation time Ti and the exhalation time Te may be estimated as typical values over many respiratory cycles of the time spent with phase Φ equal to 0 (indicating inspiration) and 0.5 (indicating expiration) respectively.

Another implementation of discrete phase determination provides a tri-valued phase output Φ with a value of one of inhalation, mid-inspiratory pause, and exhalation.

In other forms, known as continuous phase determination, the phase output Φ is a continuous variable, for example varying from 0 to 1 revolutions, or 0 to 2 π radians. RT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:

1. If Qr is zero and increasing fast then Φ is 0 revolutions.

2. If Qr is large positive and steady then Φ is 0.25 revolutions.

3. If Qr is zero and falling fast, then Φ is 0.5 revolutions.

4. If Qr is large negative and steady then Φ is 0.75 revolutions.

5. If Qr is zero and steady and the 5-second low-pass filtered absolute value of Qr is large then Φ is 0.9 revolutions.

6. If Qr is positive and the phase is expiratory, then Φ is 0 revolutions.

7. If Qr is negative and the phase is inspiratory, then Φ is 0.5 revolutions.

8. If the 5-second low-pass filtered absolute value of Qr is large, Φ is increasing at a steady rate equal to the patient's breathing rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the phase Φ is first discretely estimated from the respiratory flow rate Qr as described above, as are the inhalation time Ti and the exhalation time Te. The continuous phase Φ at any instant may be determined as the half the proportion of the inhalation time Ti that has elapsed since the previous trigger instant, or 0.5 revolutions plus half the proportion of the exhalation time Te that has elapsed since the previous cycle instant (whichever instant was more recent).

### 5.4.3.2.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template π(Φ).

In one form of the present technology, a waveform determination algorithm 4322 provides a waveform template π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template π( ) is a square-wave template, having a value of 1 for values of phase up to and including 0.5 revolutions, and a value of 0 for values of phase above 0.5 revolutions. In one form, suitable for continuously-valued phase, the waveform template π(Φ) comprises two smoothly curved portions, namely a smoothly curved (e.g. raised cosine) rise from 0 to 1 for values of phase up to 0.5 revolutions, and a smoothly curved (e.g. exponential) decay from 1 to 0 for values of phase above 0.5 revolutions. In one form, suitable for continuously-valued phase, the waveform template π(Φ) is based on a square wave, but with a smooth rise from 0 to 1 for values of phase up to a “rise time” that is less than 0.5 revolutions, and a smooth fall from 1 to 0 for values of phase within a “fall time” after 0.5 revolutions, with a “fall time” that is less than 0.5 revolutions.

In some forms of the present technology, the waveform determination algorithm 4322 selects a waveform template π(Φ) from a library of waveform templates, dependent on a setting of the RT device. Each waveform template π(Φ) in the library may be provided as a lookup table of values II against phase values Q. In other forms, the waveform determination algorithm 4322 computes a waveform template π(Φ) “on the fly” using a predetermined functional form, possibly parametrised by one or more parameters (e.g. time constant of an exponentially curved portion). The parameters of the functional form may be predetermined or dependent on a current state of the patient 1000.

In some forms of the present technology, suitable for discrete bi-valued phase of either inhalation (Q=0 revolutions) or exhalation (Q=0.5 revolutions), the waveform determination algorithm 4322 computes a waveform template π“on the fly” as a function of both discrete phase Φ and time t measured since the most recent trigger instant. In one such form, the waveform determination algorithm 4322 computes the waveform template π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - {T}_{i} ) , & Φ = 0.5\end{matrix}$$

where πᵢ(t) and πₑ(t) are inspiratory and expiratory portions of the waveform template π(Φ, t). In one such form, the inspiratory portion π(t) of the waveform template is a smooth rise from 0 to 1 parametrised by a rise time, and the expiratory portion Ile (t) of the waveform template is a smooth fall from 1 to 0 parametrised by a fall time.

### 5.4.3.2.3 Ventilation Determination

In one form of the present technology, a ventilation determination algorithm 4323 receives an input a respiratory flow rate Qr, and determines a measure indicative of current patient ventilation, Vent.

In some implementations, the ventilation determination algorithm 4323 determines a measure of ventilation Vent that is an estimate of actual patient ventilation. One such implementation is to take half the absolute value of respiratory flow rate, Qr, optionally filtered by low-pass filter such as a second order Bessel low-pass filter with a corner frequency of 0.11 Hz.

In other implementations, the ventilation determination algorithm 4323 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate shape is constant.

### 5.4.3.2.4 Determination of Inspiratory Flow Limitation

In one form of the present technology, the central controller 4230 executes an inspiratory flow limitation determination algorithm 4324 for the determination of the extent of inspiratory flow limitation.

In one form, the inspiratory flow limitation determination algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified by a zero-crossing detector. A number of evenly spaced points (for example, sixty-five), representing points in time, are interpolated by an interpolator along the inspiratory flow rate-time curve for each breath. The curve described by the points is then scaled by a scalar to have unity length (duration/period) and unity area to remove the effects of changing breathing rate and depth. The scaled breaths are then compared in a comparator with a pre-stored template representing a normal unobstructed breath, similar to the inspiratory portion of the breath shown in FIG. 7A. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by the central controller 4230 for the preceding several inspiratory events. This is repeated over the same inspiratory events for the second such point, and so on. Thus, for example, sixty-five scaled data points are generated by the central controller 4230, and represent a moving average of the preceding several inspiratory events, e.g., three events. The moving average of continuously updated values of the (e.g., sixty-five) points are hereinafter called the” scaled flow rate “, designated as Qs (t). Alternatively, a single inspiratory event can be utilised rather than a moving average.

From the scaled flow rate, two shape factors relating to the determination of” partial obstruction may be calculated.

Shape factor 1 is the ratio of the mean of the middle (e.g. thirty-two) scaled flow rate points to the mean overall (e.g. sixty-five) scaled flow rate points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical patient.

Shape factor 2 is calculated as the RMS deviation from unit scaled flow rate, taken over the middle (e.g. thirty-two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can be other than those described.

### 5.4.3.2.5 Determination of Apneas and Hypopneas

In one form of the present technology, the central controller 4230 executes an apnea/hypopnea determination algorithm 4325 for the determination of the presence of apneas and/or hypopneas.

In one form, the apnea/hypopnea determination algorithm 4325 receives as an input a respiratory flow rate signal Qr and provides as an output a flag that indicates that an apnea or a hypopnea has been detected.

In one form, an apnea will be said to have been detected when a function of respiratory flow rate Qr falls below a flow rate threshold for a predetermined period of time. The function may determine a peak flow rate, a relatively short-term mean flow rate, or a flow rate intermediate of relatively short-term mean and peak flow rate, for example an RMS flow rate. The flow rate threshold may be a relatively long-term measure of flow rate.

In one form, a hypopnea will be said to have been detected when a function of respiratory flow rate Qr falls below a second flow rate threshold for a predetermined period of time. The function may determine a peak flow, a relatively short-term mean flow rate, or a flow rate intermediate of relatively short-term mean and peak flow rate, for example an RMS flow rate. The second flow rate threshold may be a relatively long-term measure of flow rate. The second flow rate threshold is greater than the flow rate threshold used to detect apneas.

### 5.4.3.2.6 Determination of Snore

In one form of the present technology, the central controller 4230 executes one or more snore determination algorithms 4326 for the determination of the extent of snore.

In one form, the snore determination algorithm 4326 receives as an input a respiratory flow rate signal Qr and provides as an output a metric of the extent to which snoring is present.

The snore determination algorithm 4326 may comprise the step of determining the intensity of the flow rate signal in the range of 30-300 Hz. Further, the snore determination algorithm 4326 may comprise a step of filtering the respiratory flow rate signal Qr to reduce background noise, e.g., the sound of airflow in the system from the blower.

### 5.4.3.2.7 Determination of Airway Patency

In one form of the present technology, the central controller 4230 executes one or more airway patency determination algorithms 4327 for the determination of the extent of airway patency.

In one form, the airway patency determination algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency determination algorithm 4327 receives as an input a respiratory flow rate signal Qr, and determines the presence or absence of a cardiogenic signal. The absence of a cardiogenic signal is taken to be an indication of a closed airway.

### 5.4.3.2.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

In some forms of the present technology, there is no target ventilation determination algorithm 4328, and the target value Vtgt is predetermined, for example by hard-coding during configuration of the RT device 4000 or by manual entry through the input device 4220.

In other forms of the present technology, such as adaptive servo-ventilation (ASV), the target ventilation determination algorithm 4328 computes a target value Vtgt from a value Vtyp indicative of the typical recent ventilation of the patient.

In some forms of adaptive servo-ventilation, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4328, the recent history is of the order of several minutes, but in any case should be longer than the timescale of Cheyne-Stokes waxing and waning cycles. The target ventilation determination algorithm 4328 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 5.4.3.2.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$Pt = A Π ( Φ , t ) + {P}_{0} \qquad ( 1 )$$

where:
A is the amplitude,
π(Φ, t) is the waveform template value (in the range 0 to 1) at the current value Φ of phase and t of time, and
P₀ is a base pressure.

If the waveform determination algorithm 4322 provides the waveform template π(Φ, t) as a lookup table of values II indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen respiratory pressure therapy mode in the manner described below.

### 5.4.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.3.4 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g. pressure, flow rate, temperature, PaO2)
Failure of a test alarm to generate a detectable alarm signal.

Upon detection of the fault condition, the corresponding algorithm 4340 signals the presence of the fault by one or more of the following:
Initiation of an audible, visual &/or kinetic (e.g. vibrating) alarm
Sending a message to an external device
Logging of the incident

### 5.4.3.5 Detection of Interface Parameters

### 5.4.3.5.1 Systems and Devices for Detecting Interface Parameters

During use, the patient interface 3000 or 3800 may move out of place and out of the nares of the patient 1000 or may be positioned incorrectly relative to the nares of patient 1000. Although the patient interface 3800 is referenced in particular below, it will be appreciated that the systems and techniques described below also apply to other types of interfaces 3000. In some examples, the patient interface 3800 may fall entirely out of the nares of the patient 1000/off of a face of the patient 1000. In other examples, one or more of the prongs 3810a, 3810b of the patient interface 3800 may become dislodged from (e.g., slip out of) the nare(s) of the patient 1000. When portions or an entirety of the patient interface 3800 are displaced, such that HFT is not being administered to the patient 1000 as intended, the HFT may be less effective. For example, the patient 1000 may receive less air/oxygen than prescribed, and/or may receive the air/oxygen for less time than prescribed.

A system 8000 for detecting and/or communicating that the interface 3800 is out of position, for evaluating/analyzing a therapy, or for evaluating patient breathing is depicted in FIG. 5A. The system 8000 may include the RT device 4000 and the patient interface 3800 (or an alternative patient interface). FIG. 5B shows further details of the patient interface 3800. The system 8000 may optionally also include an external device 8030.

As shown in FIG. 5A, one or more of the patient interface 3800 or the RT device 4000 may include one or more sensors 8010 or another type of transducer, such as any of the transducers discussed above for RT device 4000. For example, RT device 4000 may include a flow rate sensor, such as flow rate sensor 4274, which may be positioned upstream of blower 4142. RT device 4000 additionally or alternatively may include an atmospheric pressure sensor, which may, for example, be used to correct for a change in air density with altitude and improve accuracy of an estimated PEEP. Sensors 8010 are shown in dashed lines in FIG. 5A because the sensor(s) 8010 may be in only one or the other (or neither) of the patient interface 3800 or the RT device 4000. In some examples, the sensor 8010 may include a pressure sensor. Alternatively or additionally, the sensor 8010 may include any other suitable type of sensor. For example, some types of sensors 8010 may measure or detect breath (e.g., via a pressure sensor, a temperature sensor (e.g., thermocouple), a humidity sensor, or capnography). Other types of sensors may measure proximity of, for example, the patient interface 3800 to an anatomy (e.g., nares) of the patient (e.g., a light sensor/emitter, temperature sensor, or sensors for measuring electrical impedance, capacitive touch, or resistive touch). Other types of sensors may also be used as, for example, secondary sensors (e.g., an accelerometer, orientation sensor, oximeter, or inertial measurement unit (“IMU”). If the sensor 8010 is disposed in/at the RT device 4000, the sensor 8010 may have any of the properties of pressure sensor 4272 or any of the other sensors discussed above. In some examples, the sensor 8010 may be positioned at or near an outlet of the blower 4142 (e.g., upstream of the humidifier 5000), at or near an outlet of the humidifier 5000, upstream of the blower 4142, at or near the inlet of patient interface 3800, or in any other suitable location.

As shown in FIG. 5B, the patient interface 3800 may include tubing 3830 that may extend from lumens 3820a, 3820b, toward the RT device 4000. The tubing may be an example of the air circuit 4170. The tubing 3830 may be connected to the lumens 3820a, 3820b at an interface 3840. The tubing 3830 may connect to the RT device 4000 via a connector 3850, such that the patient interface 3800 is in fluid communication with an outlet of RT device 4000. Air and/or oxygen may flow along a flow path through the RT device 4000, through the tubing 3830, and through the patient interface 3800. In some aspects, the tubing 3830 may be heated. The tubing 3830 may have varying sizes.

As show in FIG. 5B, in some examples, the sensor 8010 may be disposed at interface 3480. However, such a position is merely exemplary. The sensor 8010 may additionally or alternatively be disposed in/on one or more of lumens 3820a, 3820b and/or one or more of prongs 3810a, 3810b. Additionally or alternatively, the sensor 8010 may be disposed in or on a bridge 3860 that extends between the prongs 3810a, 3810b, at the connector 3850, or in/on the length of the tubing 3830. The patient interface 3800 also may include a sensor 8020, in addition or in the alternative to the sensor 8010. As shown in FIG. 5B, the sensor 8020 may be disposed in/on one or both of the prongs 3810a, 3810b. Alternatively or additionally, the sensor 8020 may be disposed at any of the locations discussed above as to the sensor 8010. The sensor 8020 may be the same type of sensor as the sensor 8010 or may be a different type of sensor. The sensor 8020 may be any type of sensor, including, but not limited to, the types of sensors listed above for the sensor 8010.

As shown in FIG. 5A, the external device 8030 may include a sensor 8040, which may have any of the properties of the sensors 8010, 8020. In an example, the external device 8030 may include a device for attaching to a patient, and the sensor 8040 may include a pulse oximeter. For example, the external device 8030 may include a clip or band for attaching to a finger of the patient 1000. Alternatively, the external device 8030 and the sensor 8040 may be configured to measure alternative qualities of the patient 1000 (e.g., heart rate, respiration rate, blood pressure, movement, etc.). The external device may include a band (e.g., wrist or chest band, an electrode, or a smart phone). Alternatively, the external device 8030 may be located in a room, and the sensor 8040 may be configured to measure room conditions. In further alternatives, the external device 8030 may be associated with another piece of medical equipment (e.g., medical equipment that delivers treatments or performs diagnostics).

### 5.4.3.5.2 Methods for Detecting Interface Parameters

Pressure measurements may be utilized to determine flow parameters of a therapy delivered to the patient 1000. For example, U.S. Patent Application Publication No. 2022/0160979, published May 26, 2022, incorporated herein by reference in its entirety, discloses systems and methods for estimating exit pressure (e.g., a pressure drop through air circuit and the patient interface 3800), respiratory flow rate, and a coupling parameter. U.S. Patent Application Publication No. 2022/0160979 also discloses that exit pressure, a flushing flow rate, and a respiratory flow rate may be utilized to output one or more therapy parameters for use by the RT device 4000. U.S. Patent Application Publication No. 2022/0160979 also discloses reporting of certain parameters by the central controller 4230. U.S. Patent Application Publication No. 2022/0160979 also includes other relevant disclosure not specifically identified above but pertinent to this disclosure. The methods described herein are not mutually exclusive. The steps of the methods may be combined in any suitable manner, and steps may be omitted or repeated as desired.

FIG. 6A depicts a flow chart of an exemplary method 9000 for detecting one or more interface parameters. In step 9010, data may be received (e.g., by the central controller 4230) from one or more of the sensors 8010, 8020, 8040. Although the central controller 4230 is referenced herein as performing the steps of method 9000, it will be appreciated that an alternative, local controller of RT device 4000 or a remote controller, such as remote external device 4286 (e.g., a cloud-based controller) may perform one or more of the steps of the method 9000.

In one example, the data may reflect a pressure at one or more locations of the RT device 4000, at one or more locations along the air circuit 4170 (e.g., along the tubing 3830), at one or more locations within the patient interface 3800, or at an outlet of the prongs 3810a, 3810b of the patient interface 3800. For example, as shown in FIG. 5B, the sensor 8010 may measure pressure at the interface 3840 where the patient interface 3800 and the tubing 3830 are coupled to one another. In an additional or alternative example, the sensor 8010 may measure pressure downstream of the humidifier 5000, at or near the outlet of the blower 4142, at or near the inlet of patient interface 3800, or any other suitable position.

In step 9020, the central controller 4230 may, using the data received in step 9010, analyze/determine whether the patient interface 3800 is mispositioned or displaced entirely. For example, the central controller 4230 may compare the measured pressure to a threshold value or a threshold range. The threshold value may be indicative of a value expected to be measured by the one or more sensors 8010 when the patient interface is properly positioned. The threshold value may depend, at least in part, on the settings of the RT device 4000 at that time (e.g., airflow setting, humidity setting, amount of oxygen being delivered (e.g., mix of air and oxygen), etc.), the size or type of patient interface worn, of other variables. The comparison of the measured pressure to the threshold value thus may indicate mispositioning if the measured pressure is different enough from the threshold value. In some aspects, the degree to which the measured pressure is different from the threshold value may correlate to the degree to which the patient interface 3800 is mispositioned and/or how the patient interface 3800 may be mispositioned.

In general, as compared to the pressure at the outlet of the blower 4142, the pressure of air and/or oxygen flowing will be lower after the humidifier 5000, and again lower after travelling through the tubing 3830 (e.g., at the interface 3840). Outside of the prong(s) 3810a, 3810b, the pressure will generally match the pressure inside the patient's nares. The flow of air and/or oxygen through the prong(s) 3810a, 3810b may be high enough to increase the pressure in the nares, as compared to the ambient pressure. The pressure drop between each successive location along the flow of air/oxygen may be characterized by the following equation: Pressure Drop=A*Flow+B*Flow 2. The pressure drop is measured in cmH₂O, and the flow is measured in L/s. The coefficient “A” is attributed to friction losses (laminar resistance), which is proportional to flow. The coefficient “B” is attributed to turbulent resistance (i.e., the Bernoulli equation), where pressure drop is proportional to flow squared. The coefficients may be determined empirically, by fitting a curve to a plot of flow versus pressure at an inlet at the air-delivery hose, upstream of the patient interface 3800 and the air-delivery hose. In some examples, a pressure sensor may be placed at numerous locations. For example, pressure sensors may be located before and after successive flow segments.

The coefficients may vary for different configurations of the RT device 4000, the humidifier 5000, the air circuit 4170 (e.g., the tubing 3830), and/or the patient interface 3800. For example, the patient interface may come in multiple sizes (e.g., small, medium, large), or the air circuit 4170 may come in different lengths and/or diameters. The humidifier 5000 may be included in the same model as the RT device 4000 (described above) or may be separately modeled as a component in series. U.S. Patent Application Publication No. 2004/0074495, published Apr. 22, 2004, further describes calculating pressure drops, and is incorporated herein by reference in its entirety. In some aspects, the impedance of the nasal cannula-type unsealed patient interface 3800 may dominate that of the air-delivery hose.

Memory 4260 (or another storage device) may have stored thereon a threshold value or a threshold range, as discussed above. For example, the threshold value may be a value of an expected reading of the sensor 8010 when it is properly positioned in the nares of the patient 1000. The threshold value may vary based on, e.g., a positioning of the pressure sensor 8010 within the system 8000. The threshold value may account for the pressure drop, described above, as air and/or oxygen travels through the RT device 4000, the air circuit 4170, and the patient interface 3800. For example, if the pressure reading from the sensor 8010 is higher/lower than the threshold, the central controller 4230 may determine that the patient interface 3800 is not properly positioned in the patient's nares. Certain values of the reading from the pressure sensor 8010 (e.g., a value that would indicate the prongs 3810a/3810b are flowing into the room instead of into the nares of the patient 1000) may indicate that the patient interface is completely displaced from a face of the patient 1000. Other values may indicate that the patient interface is partially displaced from the face of the patient 1000.

In some aspects, the threshold utilized may be adjustable/customizable. For example, the threshold may be set for a particular patent so as to only determine when the patient interface 3800 is completely off of the face of the patient 1000. Alternatively, the threshold may be set so as to determine when the patient interface 3800 is slightly out of position, moderately out of position, etc.

Readings obtained from multiple pressure sensors 8010, 8020, 8040 may obtained and combined, compared, correlated, filtered, or otherwise analysed during the step 9020. For example, multiple sensors of a same type (e.g., multiple sensors 8010) may be disposed in different locations of, for example, the RT device 3000, the patient interface 3800, or the air circuit 4170. Alternatively or additionally, multiple types of sensors may be utilized. The readings from these different sensors may be combined, synthesized, filtered, or otherwise analysed or calibrated. For example, stored thresholds (e.g., values, ranges, waveforms, etc.) may account for the readings of multiple sensors, or the readings from multiple sensors may be calibrated before comparing them to the threshold.

Step 9020 also may include accounting for a breath cycle of the patient 1000. At some points during a breath cycle of the patient 1000 (e.g., an exhale), a pressure drop may be smaller. At other points, such as during an inhale, a pressure drop may be larger. The central controller 4230 may be programmed to determine at what point during a breath cycle a measurement from sensor 8010 was obtained. Additionally or alternatively, step 9010 may include obtaining a plurality of measurements from the sensor 8010, and step 9020 may include synthesizing the plurality of measurements in order to account for the breath cycle. Additionally or alternatively, step 9020 may include filtering data received in step 9010 to account for a breath cycle/breath pattern of the patient 1000. Furthermore, step 9020 may include one or more of compensating for nasal cycle, whether a patient 1000 is sedentary, active, or sleeping, the prognosis of the patient 1000, a position of the patient 1000 (e.g., sitting, lying down, standing, moving), or whether a patient 1000 is talking, coughing, eating, drinking, or performing other actions with their mouth or nose. As discussed in further detail below with respect to a method 9100 (FIG. 6C), aspects of a patient's breathing (e.g., the breath cycle of the patient 1000) may be used to calibrate the threshold or adjust analysis of the measurements received in step 9010.

Additionally or alternatively, step 9020 may include comparing a breathing waveform to an expected threshold breathing waveform. For example, a typical breathing waveform may look like the waveform of FIG. 7A.

FIG. 7B shows an exemplary waveform of pressure measured versus time over the course of a breath cycle. During a time of exhalation (Te), a pressure measured by a sensor may be higher than a pressure measured during a time of inhalation (Ti). Amplitude, period/frequency, periodicity, slopes, component waveforms, or other aspects of a patient's breathing waveform may be found to differ from an expected waveform and/or plot of measured pressures if the patient interface 3800 is slightly out of position, moderately out of position, etc. For example, if the patient interface 3800 is out of position, a plot of pressure vs. time, such as that shown in FIG. 7B, may have a lower amplitude (corresponding to a maximum pressure (Pₘₐₓ) and/or a minimum pressure (Pₘᵢₙ)) than when the patient interface 3800 is properly positioned. If patient interface 3800 is off of the patient, the plot of pressure vs. time may be a flat line. As used herein, the term “threshold” may encompass at least a single threshold value, a threshold range, an expected/threshold breathing waveform, a graph, and/or a pattern. For example, a threshold may include an amplitude of a pressure waveform, such as that shown in FIG. 7B. Step 9020 may include comparing a pressure waveform representing pressure data from the sensor 8010 (and/or another sensor, such as the sensors 8020, 8040) to a threshold value. In some examples, the threshold may be a threshold amplitude and the comparison may be between an amplitude of the threshold waveform relating to the received data to the threshold amplitude. An amplitude of the pressure waveform received from the sensors 8010, 8020, and/or 8040 may be lower than the threshold if the patient interface 3800 is out of place. The amplitude of the pressure waveform may be an average amplitude across multiple breath cycles or may be obtained from a single breath cycle. The amplitude may be determined from a maximum pressure Pₘₐₓ, a minimum pressure Pₘᵢₙ, or a combination of Pₘₐₓ and Pₘᵢₙ values.

Some variation in amplitude, period/frequency, periodicity, slopes, component waveforms, or other aspects of a patient's breathing waveform may occur normally from one breath to another. The comparison of step 9020 may account for natural variation in individual breaths and may compare the general trends related to the rise and fall pattern of breath. In the example above, the general amplitude trends of the rises and falls of a patient's breath may be used as an indicator of the positioning of the patient interface 3800. In some aspects, certain patterns of the waveform may be used to refine the detection of the positioning of the patient interface 3800.

In step 9030, an alarm may be provided to the patient 1000 and/or a medical/healthcare provider upon determining that the patient interface 3800 is out of a desired position (e.g., based on the comparison(s) of step 9020). For example, output device 4290 may provide such an alarm. The alarm may be visual, auditory, tactile, or a combination thereof. The alarm may, for example, alert the patient 1000 that the patient interface 3800 is out of place and needs to be put on correctly. The alarm may be qualitative (e.g., may inform the patient 1000 that the patient interface 3800 is not positioned well and needs to be re-positioned, e.g., in a particular way) or may include a light, a sound, a vibration, or a text message. The alarm may provide a different alert depending on the positioning issue and/or the action that the patient needs to take or may provide the same alert regardless of the positioning issue and/or the action that needs to be taken. The alarm additionally or alternatively may be transmitted to a remote device via data communication interface 4280. For example, the alarm may be transmitted to a patient's device, such as a computer or smart phone, or to a health care provider's device. In aspects, the step 9030 may include displaying a dashboard to a health care provider (e.g., a physician, nurse, therapist, etc.), which may, for example, provide points of contact to the provider. The step 9030 also may include sending reports regarding positioning over time. Such reports may indicate, for example, a percentage of time during which the patient interface 3800 is appropriately positioned, and/or an analysis of patterns (e.g., time of day, patient position, patient activity level, etc.) as they relate to positioning of the patient interface 3800. Step 9030 also may include comparing the positioning data for the patient interface 3800 to a prescribed usage of the RT device 4000 and the patient interface 3800. Data obtained in step 9010 may be time-stamped, and reports may provide detailed feedback on patient compliance, positioning of the patient interface 3800, the breathing patterns of the patient 1000, etc.

In the step 9030, if a deterioration in breathing is detected that goes beyond an indication for adjustment, then an alarm may indicate that the patient should consult a physician, go to a hospital, contact a health care provider or other party, call for emergency aid, or otherwise communicate that the patient needs medical assistance. Additionally or alternatively, the system 8000 may automatically call an emergency number, contact a physician or designated emergency contact, or otherwise take automatic action.

The discussion above includes examples in which the data collected is from the sensor 8010 and the sensor 8010 is a pressure sensitive. The method 9000 also may include gathering data from other sensors, such as the sensor 8020 (further details about the use of the sensor 3840 are provided below). The sensor 8020 may also be a pressure sensor. Alternatively, the sensor 8020 may be a different kind of sensor. For example, the sensor 8020 may be a light sensor positioned on the prong 3810b, as shown in FIG. 5B. A light emitter (not shown) may be positioned on the other prong 3810a. The light sensor may determine whether or not the prongs 3810a, 3810b are positioned in the nares, based on the amount of light measured by the sensor 8020. For example, if an amount of light measured by the sensor 8020 exceeds a threshold, in step 9020, the central controller 4230 may determine that the prongs 3810a, 3810b are not properly positioned within the nares of the patient 1000. Additionally or alternatively, a light emitter may transmit light through tissue, and the sensor 8020 may measure/detect a cardiac pulse based on absorption of the light in order to determine whether the prongs 3810a, 3810b are properly positioned or to determine other aspects of treatment delivery, efficacy, etc. In other alternatives, the sensor 8020 may measure other parameters (e.g., temperature, humidity, position, flow rate, etc.) that may be indicative of whether the patient interface 3800 is positioned properly.

The steps of the method 9000 may be repeated periodically (e.g., at set intervals), at irregular intervals, continuously, after certain phases of treatment, at certain amounts of time during the treatment, or after a patient adjusts the treatment (e.g., to check if the adjustment was made as the result of patient interface 3800 being mispositioned). Step 9030 may include providing one or more progress reports showing how the positioning of the patient interface 3800 has changed over time (e.g., across minutes, hours, days, weeks, months, or years).

FIG. 6B depicts another method 9050, which may allow for control of RT device 4000. The method 9050 may utilize any of the steps of the method 9000, as described above. In step 9060, the central controller 4230 may receive data from one or more sensor(s). The step 9060 may include receiving any data described above. Additionally, the step 9060 may include receiving data from the sensor 8040 of the external device 8030. The sensor 8040 may include a sensor that obtains data that is indicative of the effectiveness of the treatment administered by the RT device 4000. In one example, the sensor 8040 may be an oximeter configured to measure a blood oxygen saturation of the patient 1000. Alternatively, the sensor 8040 may be configured to measure movements of the patient (e.g., the sensor 8040 may include an IMU, accelerometer, position sensor, heart rate sensor, etc.). In some aspects, decreased activity levels of the patient 1000 may be indicative of a lower effectiveness of the treatment. The sensor 8040 may alternatively measure other types of data.

In step 9070, the central controller 4230 may evaluate/analyze the effectiveness of a treatment with the RT device 4000. The step 9070 may include determining whether the data from the sensor 8040 shows an adequate efficacy of the therapy (e.g., by comparing the data from the sensor 8040 to the threshold (e.g., a threshold value, threshold range, or threshold waveform). The step 9070 may incorporate some or all of the analyses of method 9000, discussed above. For example, the step 9070 may account for an amount of time that the patient interface 3800 is appropriately positioned. If the central controller 4230 determines that the patient interface 3800 is appropriately positioned for an acceptable amount of time (e.g., above a threshold of an amount of time or a percentage of time) and that the therapy fails to have adequate efficacy (e.g., because the data from the sensor 8040 is above/below a threshold), then the step 9070 may determine that the prescribed therapy is inadequate. In other words, the central controller 4230 may determine that, despite compliance and proper positioning of the patient interface 3800, the prescribed therapy is not providing the intended results. Alternatively, if the step 9070 determines that the patient interface 3800 is not properly positioned for an adequate amount of time (e.g., is mispositioned for too great of a time or a percentage of time), then the step 9070 may determine that the efficacy of the prescribed therapy cannot be assessed. The output device 4290 (or another device) or a medical professional may inform the patient 1000 that the patient interface 3800 is not being worn properly and may instruct the patient 1000 on proper wearing (or may prescribe a different patient interface 3000 or 3800). In a further alternative, the central controller 4230 may determine that an adjustment to the therapy may compensate for improper positioning of the patient interface 3800.

Depending on the above, in the step 9080, the central controller 4230 may determine whether adjustments to a prescribed therapy are required. For example, in step 9080, the central controller 4230 may determine that adjustments are required to a flow rate of the air and/or oxygen, amount of oxygen delivered (e.g., mix of air/oxygen), amount of time for the patient interface to be worn, type of patient interface to be worn, size of patient interface to be worn, etc., in order to address any lack of efficacy based on the analysis of the data from the sensor 8040 and/or the sensors 8010, 8020. Alternatively or additionally, the central controller 4230 may inform a health care provider of the analysis. Step 9080 may, in some aspects, include determining the adjustments to be made.

If adjustments are required, in a step 9090, a parameter of the therapy may be adjusted based on the analysis of the steps 9070 and/or 9080 (e.g., using the therapy device controller 4240). For example, a flow rate, or amount of oxygen delivered (e.g., mix of air/oxygen) may be altered. The step 9090 may be performed automatically, based on the analysis of the steps 9070 and/or 9080, such that they system 8000 forms a closed-loop system. Alternatively, the step 9090 may be performed manually, e.g., by a patient or a health care provider. Alternatively, if, in step 9080, adjustments are determined not to be required, then the method 9050 may terminate in step 9095. The steps of the method 9050 may be repeated periodically (e.g., at set intervals), continuously, at irregular intervals, after certain phases of treatment, at certain amounts of time during the treatment, or after a patient adjusts the treatment (e.g., to check if the adjustment taken was the result of patient interface 3800 being mispositioned). In some aspects, the steps of method 9050 may be performed more frequently if it is decided in step 9080 that adjustments are indicated, e.g., to assess whether the instituted adjustments are working.

As discussed above with respect to the step 9030, the step 9090 also may include alerting a patient to seek medical attention or automatically contacting a physician or other party to obtain medical assistance if a patient's breathing deteriorates to a designated extent (e.g., beyond a threshold).

The thresholds (e.g., values, ranges, or waveforms) used in steps 9020 and 9070 may be patient-specific. For example, method 9100 of FIG. 6C includes a step 9110 of receiving baseline data for a patient 1000 from one or more of the sensors 8010, 8020, 8040 (or another sensor), and/or prior patient history data. The baseline data may be obtained at a time when the patient 1000 is breathing normally or from long-term averages expected to reflect normal breathing. In examples, the baseline data may be obtained when the patient interface 3800 is positioned correctly/ideally. Additional baseline data may be obtained when the patient interface 3800 is out of position in one or more configurations (e.g., baseline data may be obtained for varying types and degrees of positioning errors). In some aspects, this type of data may be collected initially when setting up a patient with an RT device 4000. In collecting the data during the step 9110, the RT device 4000 may perform a set of particular actions in order to collect the desired data. For example, the memory 4260 may store data collection/calibration protocol(s) to be performed by the central controller 4230. For example, the RT device 4000 may deliver air and/or oxygen at different parameters (e.g., different flow amounts, different air/oxygen mixes, etc.), and data may be collected from the sensors 8010, 8020, 8040 at the different parameters. In step 9110, data regarding an environment also may be collected. For example, data regarding a patient's location, whether conditions, altitude, ambient pressure, or pollution levels at that location may be collected.

In step 9120, the baseline data collected in step 9110 may be utilized to generate patient-specific thresholds (e.g., values, ranges, patterns, graphs, and/or waveforms). The thresholds may be any of the types of thresholds discussed above for methods 9000 and 9050. For example, the threshold may be a pressure threshold that would indicate a pressure at an ideal position of the patient interface 3800 and various pressures for different positioning errors. Additionally or alternatively, other types of thresholds or patterns (e.g., breathing patterns, variations between inhalations and exhalations, etc.) may be generated. For example, typical, baseline breathing rates, inhalation/exhalation volumes, or other parameters may be collected.

Curves/graphs/waveforms may be generated that reflect the baseline breathing pattern of the patient 1000.

In step 9130, the thresholds and/or patterns generated in step 9120 may be stored in, for example, memory 4260. The stored thresholds and/or patterns may be utilized in, for example, steps 9020, 1970, and/or 9080 of methods 9000 and/or 9050.

In some examples, method 9100 may be repeated at regular intervals (e.g., during regular wear by a patient 1000 or after prompting the patient 1000 to begin a calibration procedure). The RT device 4000 or a patient interface on another device may prompt the patient 1000 to position the patient interface 3800 in a particular way and/or to perform certain steps on RT device or another interface. When method 9100 is performed/repeated, a user interface may prompt the patient 1000 to input data (e.g., how sick/well the patient is feeling, whether the patient has a respiratory infection or allergies, whether the patient has been smoking, environmental details, etc.). In this way, the obtained baseline information may be calibrated based on, at least in part, other factors that may affect a patient's breathing and thus the information detected by the one or more sensors.

In some examples, machine learning may be utilized to perform the step 9120. For example, population-based data or patient-specific data (or a combination thereof) may be used to train the machine learning model. Step 9110 may be repeated at a later date, and the trained machine learning model may be used to update the thresholds/patterns in step 9120. In some aspects, over time, the machine learning algorithm may be used to develop a model of the breathing patterns/characteristics of the patient 1000. The machine learning algorithm also may be utilized in order to characterize the breathing of the patient 1000 under different conditions (e.g., when the patient has a respiratory infection or allergies, when the weather conditions vary, when pollution is better/worse, when at different altitudes, etc.).

FIG. 6D depicts an exemplary method 9200 for utilizing the patient-specific thresholds generated in method 9100. Methods 9000 and 9050 also may utilize the patient-specific thresholds (e.g., in steps 9020, 9070, 9080). In particular, any aspects of step 9220, described below, may be utilized in steps 9020, 9070, 9080.

In a step 9210, data may be received from one or more sensor(s) 8010, 8020, 8040. Step 9210 may incorporate any of the aspects of steps 9010, 9060, 9110, described above.

In step 9220, the data obtained in step 9210 may be compared to the patient-specific thresholds generated in method 9100 in order to characterize/analyze a positioning (e.g., correct positioning or incorrect positioning) of the patient interface 3800. Additionally or alternatively, the data obtained in step 9210 may be compared to thresholds and/or other data about aspects of the breathing of the patient 1000 (e.g., curves or graphs of the breathing of the patient 1000). The analysis of the step 9220 may be used to characterize aspects of the breathing of the patient 1000. For example, the analysis of the step 9220 may be used to evaluate whether the patient 1000 is congested, is coughing (characterized by a short, percussive burst), is smoking, or is located in an environment having a particular temperature, humidity, pollution level, altitude, or other quality. The analysis also may determine a position of the patient 1000 or characterize a progression (or lack thereof) of a disease of the patient 1000. Step 9220 also may include evaluating the therapy being provided, as described for step 9070 or method 9050.

In step 9230, an alarm or other update may be provided to the patient 1000 or to a health care provider. The alarm or other update may be provided upon determining that such an alarm/update is required. As discussed above, the alarm or information may be provided in any suitable manner (e.g., via a auditory or visual signal, via text on a user interface, or via a provider dashboard). Additionally or alternatively, the analysis may be used to adjust a therapy, as described in the steps 9080, 9090 of method 9050. As discussed above, with respect to step 9030, step 9230 may include notifying the patient 1000 to obtain health care assistance if breathing deteriorates to a defined extent and/or automatically notifying a physician or emergency medical personnel.

The methods above may be performed at any suitable interval. For example, the methods 9000 and 9200 may be performed at short intervals (e.g., continuously or almost continuously). Alternatively, longer intervals may be used. A length of interval may be customized for a particular patient 1000 and/or may vary based on any suitable variables (e.g., time of day, location of the patient 1000, parameters of the therapy being administered, position of the patient 1000, or any other relevant parameter). One or more of methods 9000, 9050, 9100, or 9200 may be performed independent from one another or in combination with one another.

### 5.5 Air Circuit

An air circuit 4170 in accordance with an aspect of the present technology is a conduit or a tube constructed and arranged to allow, in use, a flow of air to travel between two components such as RT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 5.6 Breathing Waveforms

FIG. 7 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6s, peak inspiratory flow rate Qpeak 0.4 L/s, exhalation time Te 2.4s, peak expiratory flow rate Qpeak-0.5 L/s. The total duration of the breath, Ttot, is about 4s. The person typically breathes at a rate of about 15 breaths per minute (BPM), with Ventilation Vent about 7.5 L/min. A typical duty cycle, the ratio of Ti to Ttot, is about 40%.

### 5.7 Respiratory Therapy Modes

Various respiratory therapy modes may be implemented by the disclosed respiratory therapy system.

### 5.7.1 High Flow Therapy

In other forms of respiratory therapy, the pressure of the flow of air is not controlled as it is for respiratory pressure therapy. Rather, the central controller 4230 controls the pressure generator 4140 to deliver a flow of air whose device flow rate Qd is controlled to a treatment or target flow rate Qtgt that is typically positive throughout the patient's breathing cycle. Such forms are generally grouped under the heading of flow therapy. In flow therapy, the treatment flow rate Qtgt may be a constant value that is hard-coded or manually entered to the RT device 4000. If the treatment flow rate Qtgt is sufficient to exceed the patient's peak inspiratory flow rate, the therapy is generally referred to as high flow therapy (HFT). Alternatively, the treatment flow rate may be a profile Qtgt (t) that varies over the respiratory cycle.

### 5.8 Glossary

For the purposes of the present technology disclosure, in certain forms of the present technology, one or more of the following definitions may apply. In other forms of the present technology, alternative definitions may apply.

### 5.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RT device or emanating from a mask or patient interface. Ambient noise may be generated by sources outside the room.

Automatic Positive Airway Pressure (ΔPAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H2O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar~ 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.8.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

5.10 REFERENCE SIGNS LIST

| | |
|---|---|
| patient | 1000 |
| bed partner | 1100 |
| patient interface | 3000 |
| vent | 3400 |
| ISO | 3744 |
| patient interface | 3800 |
| prongs | 3810a |
| prongs | 3810b |
| lumens | 3820a |
| lumens | 3820b |
| tubing | 3830 |
| interface | 3840 |
| connector | 3850 |
| bridge | 3860 |
| RT device | 4000 |
| external housing | 4010 |
| upper portion | 4012 |
| portion | 4014 |
| panel | 4015 |
| chassis | 4016 |
| handle | 4018 |
| pneumatic block | 4020 |
| air filter | 4110 |
| inlet air filter | 4112 |
| outlet air filter | 4114 |
| muffler | 4120 |
| inlet muffler | 4122 |
| outlet muffler | 4124 |
| pressure generator | 4140 |
| blower | 4142 |
| motor | 4144 |
| anti - spill back valve | 4160 |
| air circuit | 4170 |
| air circuit | 4171 |
| supplementary gas | 4180 |
| electrical components | 4200 |
| single Printed Circuit Board Assembly | 4202 |
| PCBA | |
| power supply | 4210 |
| input device | 4220 |
| central controller | 4230 |
| clock | 4232 |
| therapy device controller | 4240 |
| protection circuits | 4250 |
| memory | 4260 |
| transducer | 4270 |
| pressure sensor | 4272 |
| flow rate sensor | 4274 |
| motor speed transducer | 4276 |
| data communication interface | 4280 |
| remote external communication network | 4282 |
| local external communication network | 4284 |
| remote external device | 4286 |
| local external device | 4288 |
| output device | 4290 |
| display driver | 4292 |
| display | 4294 |
| algorithms | 4300 |
| pre - processing module | 4310 |
| interface pressure algorithm | 4312 |
| vent flow rate estimation algorithm | 4314 |
| leak flow rate estimation algorithm | 4316 |
| respiratory flow rate estimation | 4318 |
| algorithm | |
| therapy engine module | 4320 |
| phase determination algorithm | 4321 |
| waveform determination | 4322 |
| ventilation determination | 4323 |
| inspiratory flow limitation determination | 4324 |
| apnea/hypopnea determination | 4325 |
| snore determination | 4326 |
| airway patency determination algorithm | 4327 |
| target ventilation determination | 4328 |
| algorithm | |
| therapy parameter determination | 4329 |
| algorithm | |
| therapy control module | 4330 |
| corresponding algorithm | 4340 |
| method | 4500 |
| step | 4520 |
| step | 4530 |
| step | 4540 |
| step | 4550 |
| step | 4560 |
| humidifier | 5000 |
| system | 8000 |
| sensor | 8010 |
| sensor | 8020 |
| device | 8030 |
| sensor | 8040 |

1. A system for analysing a high flow respiratory therapy, comprising:
a blower;
a nasal cannula, wherein a flow of gas travels from the blower to the nasal cannula along a flow path during administration of the high flow respiratory therapy;
a sensor disposed along the flow path; and
a processor configured to perform steps to:
receive data from the sensor;
compare the data received from the sensor to a threshold value;
based at least in part on the comparison, determine whether the nasal cannula is mispositioned on a patient or displaced from the patient; and
upon determining the nasal cannula is mispositioned on the patient or displaced from the patient, transmit an alert indicating that the nasal cannula is mispositioned on the patient or displaced from the patient.

2. The system of claim 1, wherein the processor and the blower are disposed within a respiratory therapy device.

3. The system according to claim 1, further comprising a tubing that is coupled to the nasal cannula at an interface, wherein the sensor is disposed at the interface.

4. The system of claim 3, wherein the sensor is a pressure sensor, and wherein the threshold value is an amplitude of a pressure waveform.

5. The system according to claim 1, wherein the threshold value is a patient-specific threshold value.

6. The system of claim 5, wherein the patient-specific threshold value is based on analysis of data from the sensor that is obtained during a calibration.

7. The system according to claim 5, wherein the patient specific threshold value is generated based on machine learning.

8. The system according to claim 1, wherein the sensor is a first sensor and the system further comprises a second sensor, the steps further comprising:
receive data from the second sensor; and
evaluate an efficacy of the respiratory therapy based at least in part on the data received from the second sensor.

9. The system according to claim 1, wherein the alert is transmitted to the patient.

10. The system according to claim 1, wherein the alert includes a report provided to a medical provider.

11. A system for analysing a high flow respiratory therapy, comprising:
a respiratory therapy device;
a nasal cannula;
a tubing that is configured to couple to the nasal cannula at an interface, wherein a flow of gas travels from the respiratory therapy device, through the tubing, and to the nasal cannula during administration of the high flow respiratory therapy;
a pressure sensor disposed in or on the nasal cannula or at the interface; and
a processor configured to perform steps to:
receive data from the pressure sensor;
analyze the data received from the pressure sensor;
based at least in part on the analysis, determine whether the nasal cannula is mispositioned on a patient or displaced from the patient by comparing an amplitude of a pressure waveform of the received data to a threshold amplitude; and
upon determining the nasal cannula is mispositioned on the patient or displaced from the patient, transmit an alert indicating that the nasal cannula is mispositioned on the patient or displaced from the patient.

12. The system of claim 11, wherein the processor is disposed within the respiratory therapy device.

13. The system according to claim 11, wherein the analysis includes comparing the data received from the pressure sensor to a patient-specific threshold value.

14. The system of claim 13, wherein the patient-specific threshold value is based on analysis of data from the pressure sensor that is obtained during a calibration.

15. The system according to claim 13, wherein the patient specific threshold value is generated based on machine learning.

16. The system according to claim 11, wherein the system includes a second sensor, the steps further comprising:
receiving data from the second sensor; and
evaluating an efficacy of the respiratory therapy from the respiratory therapy device based at least in part on the data received from the second sensor.

17. The system according to claim 11, wherein the system includes a second sensor, and the second sensor is a different type of sensor than the pressure sensor.

18. The system according to claim 11, wherein the alert is transmitted to the patient.

19. The system according to claim 11, wherein the alert includes a report provided to a medical provider.

20. A system for analysing a high flow respiratory therapy, comprising:
a blower;
a nasal cannula, wherein a flow of gas travels from the blower to the nasal cannula along a flow path during administration of the high flow respiratory therapy;
a pressure sensor disposed along the flow path; and
a processor configured to perform steps to:
receive data from the pressure sensor;
compare an amplitude of a pressure waveform of the data received from the pressure sensor to a patient-specific threshold value;
based on the comparison, analyze a positioning of the nasal cannula or an aspect of a patient's breathing; and
transmit an alert to the patient based on the analysis when the analysis indicates incorrect positioning of the nasal cannula or a deviation in the aspect of the patient's breathing.
